(19) United States
(12) Patent Application Publication
(10) Pub. No.: US 2026/0023085 A1
(43) Pub. Date: Jan. 22, 2026

(54) COMPOSITIONS AND METHODS FOR ANALYSIS, DETECTION, AND TREATMENT OF MULTIPLE SCLEROSIS

(71) Applicant: The Regents of the University of Michigan, Ann Arbor, MI (US)

(72) Inventors: Qi Wu, Ann Arbor, MI (US); Yang Mao-Draayer, Ann Arbor, MI (US)

(21) Appl. No.: 18/843,758

(22) PCT Filed: Mar. 14, 2023
(86) PCT No.: PCT/US2023/015212

§ 371 Date: Sep. 4, 2024

Related U.S. Application Data
(60) Provisional application No. 63/319,501, filed on Mar. 14, 2022.

(51) Int. Cl. G01N 33/68

(52) U.S. Cl. CPC G01N 33/6896; G01N 2333/485; G01N 2333/523; G01N 2333/5255; G01N 2333/5434; G01N 2333/5437; G01N 2333/57; G01N 2333/70578; G01N 2800/285; G01N 2800/56

(57) ABSTRACT

The present disclosure relates to materials and methods for prognostic classification of multiple sclerosis patients. In particular, provided herein are biomarkers and methods of uses thereof for stratifying patients with multiple sclerosis. The methods described herein facilitate selection of an appropriate treatment for a subject.

### STATEMENT REGARDING RELATED APPLICATIONS

This application is a national phase application under 35 U.S.C. § 371 of PCT International Application No.: PCT/US2023/015212, filed on Mar. 14, 2023, which claims the benefit of U.S. Provisional Patent Application No. 63/319,501, filed Mar. 14, 2022, the entire contents of which are incorporated herein by reference for all purposes.

### STATEMENT REGARDING FEDERALLY SPONSORED RESEARCH OR DEVELOPMENT

This invention was made with government support under A1110557 and AI144298 awarded by the National Institutes of Health. The government has certain rights in the invention.

### FIELD

The present disclosure relates to materials and methods for prognostic classification of multiple sclerosis patients. In particular, provided herein are biomarkers and methods of uses thereof for stratifying patients with multiple sclerosis. The methods described herein facilitate selection of an appropriate treatment for a subject.

### BACKGROUND

Multiple sclerosis (MS) is an autoimmune disease associated with the central nervous system (CNS). The pathology of MS is characterized by T- and B-lymphocyte and innate immune cell infiltration into the CNS, myelin sheath breakdown, oligodendrocyte damage, and axonal degeneration. The causes of MS have not been fully elucidated, although certain genes and environmental factors increase susceptibility. MS typically presents between the ages of 20 and 40 years. The disease phenotype is heterogeneous and varies from person to person in terms of disease trajectory and progression. In most cases, relapsing-remitting MS (RRMS) progresses to secondary progressive MS (SPMS) indicated by a steady increase in long term disability and disease severity. In less than 5-10% of cases, relapses are mild, and disease progression does not occur. This is known as non-progressive benign MS (NPMS; BMS), where individuals are functionally unaffected in terms of disability progression. As non-progressive BMS is typically diagnosed with 15-20 years of disease duration by the lack of progression without the use of disease-modifying therapies (DMTs), those diagnosed with BMS and SPMS tend to be naturally matched for age and disease duration, and the only major discernable difference is the rate of progression.

There is currently a poor understanding of the mechanisms underlying disease progression, which has made it difficult to develop effective therapies for progressive MS. Accordingly, there is a need for successful identification of specific markers to predict MS disease progression, which has the potential to uncover biological drivers of progression and targets for therapeutic intervention. Plasma markers could also help to stratify individuals according to the rate of progression, allowing for better tailoring of clinical treatment plans. The present invention addresses this need.

### SUMMARY

In some aspects, provided herein is a method comprising measuring levels of soluble CD40L (sCD40L), Monocyte Chemoattractant Protein-1 (MCP-1) and gamma interferon (IFNγ) in a sample obtained from a subject having or suspected of having multiple sclerosis (MS). In some embodiments, the sample is a plasma sample. In some embodiments, the levels of soluble CD40L (sCD40L), Monocyte Chemoattractant Protein-1 (MCP-1) and gamma interferon (IFNγ) are measured by an immunoassay.

In some aspects, provided herein is a method of stratifying multiple sclerosis in a subject. In some embodiments, the method comprises measuring levels of soluble CD40L (sCD40L), Monocyte Chemoattractant Protein-1 (MCP-1) and gamma interferon (IFNγ) in a sample obtained from a subject having or suspected of having multiple sclerosis. In some embodiments, the method comprises identifying the subject as having relapsing-remitting multiple sclerosis when the level of MCP-1 in the sample is not elevated compared to a cutoff value. In some embodiments, the method comprises identifying the subject as having progressed from relapsing-remitting multiple sclerosis to secondary progressive multiple sclerosis when the level of sCD40L in the sample is elevated compared to a cutoff value and the level of MCP-1 in the sample is elevated compared to a cutoff value. In some embodiments, the method comprises identifying the subject as having progressed from relapsing-remitting multiple sclerosis to secondary progressive multiple sclerosis when the level of sCD40L in the sample is elevated compared to a cutoff value and the level of IFNγ in the sample is elevated compared to a cutoff value. In some embodiments, the method comprises identifying the subject as having non-progressive benign multiple sclerosis when the level of MCP-1 in the sample is elevated compared to a cutoff value and the level of sCD40L in the sample is not elevated compared to a cutoff value. In some embodiments, the method comprises identifying the subject as having non-progressive benign multiple sclerosis when the level of MCP-1 in the sample is elevated compared to a cutoff value and the level of IFNγ in the sample is not elevated compared to a cutoff value. In some embodiments, the sample is a plasma sample.

In some embodiments, provided herein is a method of stratifying multiple sclerosis in a subject, comprising measuring levels of soluble CD40L (sCD40L), Monocyte Chemoattractant Protein-1 (MCP-1) and/or gamma interferon (IFNγ) in a plasma sample obtained from a subject having or suspected of having multiple sclerosis. In some embodiments, the method comprises identifying the subject as having relapsing-remitting multiple sclerosis when the level of MCP-1 in the sample is less than 369.4 pg/mL. In some embodiments, the method comprises identifying the subject as having progressed from relapsing-remitting multiple sclerosis to secondary progressive multiple sclerosis when the level of sCD40L in the sample is greater than 487.6 pg/mL and the level of MCP-1 in the sample is greater than 369.5 pg/mL, or the level of sCD40L in the sample is greater than 2206 μg/mL and the level of IFNγ in the sample is greater than 7.805 pg/mL. In some embodiments, the method comprises identifying the subject as having non-progressive benign multiple sclerosis when the level of MCP-1 in the sample is greater than 371.9 pg/mL and the level of sCD40L is less than 2206 μg/mL, or the level of MCP-1 in the sample is greater than 371.9 pg/mL and the level of IFNγ in the sample is less than 7.806 pg/mL.

In some embodiments, provided here in is a method comprising measuring levels of soluble CD40L (sCD40L) and Monocyte Chemoattractant Protein-1 (MCP-1) in a sample obtained from a subject having or suspected of having multiple sclerosis. In some embodiments, the method further comprises identifying the subject as having relapsing-remitting multiple sclerosis when the level of MCP-1 in the sample is not elevated compared to a cutoff value; or progressed from relapsing-remitting multiple sclerosis to secondary progressive multiple sclerosis when the level of MCP-1 in the sample is elevated compared to a cutoff value and the level of sCD40L in the sample is elevated compared to a cutoff value. In some embodiments, the sample is a plasma sample. In some embodiments, the cutoff value for MCP-1 is 369.4 pg/mL. In some embodiments, the cutoff value for sCD40L is 487.6 pg/mL.

In some embodiments, provided herein is a method comprising measuring a level of Monocyte Chemoattractant Protein-1 (MCP-1) in a sample obtained from a subject having or suspected of having multiple sclerosis. In some embodiments, the method further comprises identifying the subject as having relapsing-remitting multiple sclerosis when the level of MCP-1 in the sample is not elevated compared to a cutoff value; or benign non-progressive benign multiple sclerosis when the level of MCP-1 in the sample is elevated compared to a cutoff value. In some embodiments, the method further comprises measuring a level of sCD40L and/or a level of IFNγ in the sample, and identifying the subject as having benign non-progressive benign multiple sclerosis when the level of MCP-1 in the sample is elevated compared to a cutoff value and: the level of sCD40L is not elevated in the sample compared to a cutoff value or the level of IFNγ in the sample is not elevated compared to a cutoff value. In some embodiments, the cutoff value for MCP-1 is 371.9 pg/mL. In some embodiments, the cutoff value for sCD40L is 2206 μg/mL and the cutoff value for IFNγ is 7.805.

In some embodiments, provided herein is a method comprising measuring levels of soluble CD40L (sCD40L) and gamma interferon (IFNγ) in a sample obtained from a subject having or suspected of having multiple sclerosis. In some embodiments, the method further comprises identifying the subject as having progressed from relapsing-remitting multiple sclerosis to secondary progressive multiple sclerosis when the level of sCD40L in the sample is elevated compared to a cutoff value and the level of IFNγ in the sample is elevated compared to a cutoff value. In some embodiments, the sample is a plasma sample. In some embodiments, the cutoff value for sCD40L is 2206 μg/mL and the cutoff value for IFNγ is 7.805.

In some embodiments, provided herein is a method comprising measuring levels of soluble Monocyte Chemoattractant Protein-1 (MCP-1), gamma interferon (IFNγ), and neurofilament light chain (NFL) in a sample obtained from a subject having or suspected of having multiple sclerosis (MS). In some embodiments, levels of MCP-1, IFNγ, and NFL are measured by an immunoassay.

In some embodiments, provided herein are methods of stratifying multiple sclerosis in a subject. In some embodiments, provided herein is a method of stratifying multiple sclerosis in a subject, comprising measuring levels of Monocyte Chemoattractant Protein-1 (MCP-1), gamma interferon (IFNγ), and at least one of soluble CD40L (sCD40L) or neurofilament light chain (NFL) in a sample obtained from a subject having or suspected of having multiple sclerosis. In some embodiments, the method comprises identifying the subject as having relapsing-remitting multiple sclerosis when the level of MCP-1 in the sample is not elevated compared to a cutoff value. In some embodiments, the method comprises identifying the subject as having progressed from relapsing-remitting multiple sclerosis to secondary progressive multiple sclerosis when the level of NFL or the level of sCD40L in the sample is elevated compared to a cutoff value and the level of MCP-1 in the sample is elevated compared to a cutoff value, or the level of IFNγ in the sample is elevated compared to a cutoff value. In some embodiments, the method comprises identifying the subject as having non-progressive benign multiple sclerosis when the level of MCP-1 in the sample is elevated compared to a cutoff value and (i) the level of sCD40L in the sample is not elevated compared to a cutoff value or (ii) the level of IFNγ in the sample is not elevated compared to a cutoff value. For example, in some embodiments the method comprises identifying the subject as having non-progressive benign multiple sclerosis when the level of MCP-1 in the sample is elevated compared to a cutoff value and the level of sCD40L in the sample is not elevated compared to a cutoff value. As another example, in some embodiments the method comprises identifying the subject as having non-progressive benign multiple sclerosis when the level of MCP-1 in the sample is elevated compared to a cutoff value and the level of IFNγ in the sample is not elevated compared to a cutoff value.

In some embodiments, provided herein is a method of stratifying multiple sclerosis in a subject, comprising measuring levels of soluble CD40L (sCD40L), neurofilament light chain (NFL), Monocyte Chemoattractant Protein-1 (MCP-1) and/or gamma interferon (IFNγ) in a plasma sample obtained from a subject having or suspected of having multiple sclerosis. In some embodiments, the method comprises identifying the subject as having relapsing-remitting multiple sclerosis when the level of MCP-1 in the sample is less than 369.4 pg/mL. In some embodiments, the method comprises identifying the subject as having progressed from relapsing-remitting multiple sclerosis to secondary progressive multiple sclerosis when: (i) the level of sCD40L in the sample is greater than 487.6 pg/mL and the level of MCP-1 in the sample is greater than 369.5 pg/mL, (ii) the level of sCD40L in the sample is greater than 2206 μg/mL and the level of IFNγ in the sample is greater than 7.805 pg/mL, (iii) the level of NFL in the sample is greater than 10 μg/mL, (iv) the level of NFL in the sample is greater than 10 μg/mL and the level of MCP-1 in the sample is greater than 369.5 pg/mL, or (v) the level of NFL in the sample is greater than 10 μg/mL and the level of IFNγ in the sample is greater than 7.805 pg/mL. In some embodiments the method comprises identifying the subject as having non-progressive benign multiple sclerosis when (i) the level of MCP-1 in the sample is greater than 371.9 pg/mL and the level of sCD40L is less than 2206 μg/mL, (ii) the level of MCP-1 in the sample is greater than 371.9 pg/mL and the level of IFNγ in the sample is less than 7.806 pg/mL, or (iii) the level of MCP-1 in the sample is greater than 371.9 pg/mL and the level of NFL in the sample is less than 10 μg/mL.

In some embodiments, provided herein is a method comprising measuring levels of Monocyte Chemoattractant Protein-1 (MCP-1) and at least one of soluble CD40L (sCD40L) or neurofilament light chain (NFL) in a sample obtained from a subject having or suspected of having multiple sclerosis. In some embodiments, the method comprises identifying the subject as having relapsing-remitting multiple sclerosis when the level of MCP-1 in the sample is not elevated compared to a cutoff value. In some embodiments, the method comprises identifying the subject as having progressed from relapsing-remitting multiple sclerosis to secondary progressive multiple sclerosis when the level of MCP-1 in the sample is elevated compared to a cutoff value and the level of at least one of sCD40L or NFL in the sample is elevated compared to a cutoff value. In some embodiments, the cutoff value for MCP-1 is 369.4 pg/mL. In some embodiments, the cutoff value for sCD40L is 487.6 pg/mL or the cutoff value for NFL is 10 μg/mL.

In some embodiments, provided herein is a method comprising measuring levels of neurofilament light chain (NFL) and at least one of soluble CD40L (sCD40L) or gamma interferon (IFNγ) in a sample obtained from a subject having or suspected of having multiple sclerosis. In some embodiments, the method comprises identifying the subject as having non-progressive benign multiple sclerosis when the level of NFL in the sample is increased compared to a cutoff value and the level of at least one of sCD40L or IFNγ is not increased compared to a cutoff value. In some embodiments, the method comprises identifying the subject as having progressed from relapsing-remitting multiple sclerosis to secondary progressive multiple sclerosis when the level of NFL in the sample is elevated compared to a cutoff value and the level of at least one of sCD40L or IFNγ) in the sample is elevated compared to a cutoff value. In some embodiments, the cutoff value for NFL is 6 pg/mL and (i) the cutoff value for sCD40L is 2206 μg/mL or (ii) the cutoff value for IFNγ is 7.805 pg/mL.

In some embodiments, provided herein is a method comprising measuring a level of neurofilament light chain (NFL) in a sample obtained from a subject having or suspected of having multiple sclerosis, and identifying the subject as having secondary progressive multiple sclerosis when the level of neurofilament light chain (NFL) in the sample is elevated compared to a cutoff value. In some embodiments, the cutoff value is 10 μg/mL.

In some embodiments, the methods described herein further comprise monitoring the subject identified as having BMS. In some embodiments, the methods described herein further comprise treating the subject identified as having relapsing-remitting multiple sclerosis with a disease-modifying therapy (DMT). In some embodiments, the DMT comprises one or more therapies selected from beta interferon, glatiramer acetate, fingolimod, dimethyl fumarate, diroximel fumarate, teriflunomide, siponimod, cladribine, a monoclonal antibody, or mitoxantrone. In some embodiments, the monoclonal antibody is selected from, ocrelizumab, natalizumab, and alemtuzumab. In some embodiments, the methods described herein further comprise treating the subject identified as having secondary progressive multiple sclerosis with one or more therapies selected from stem cell therapy, steroids, beta interferon, cladribine, and Siponimod. In some embodiments, the subject is human. In some embodiments, levels of soluble CD40L (sCD40L), neurofilament light chain (NFL), Monocyte Chemoattractant Protein-1 (MCP-1) and/or gamma interferon (IFNγ) are measured by an immunoassay.

### DESCRIPTION OF THE DRAWINGS

FIG. 1 shows a Z-score heatmap comparing relative expression levels of different soluble factors detected in plasma among HC and 3 types of MS. Expression levels of 8 significantly different (P<0.05) plasma soluble factors detected using Luminex and analyzed with Kruskal-Wallis test comparing HC, RRMS, BMS, and SPMS are displayed after Z-transformation. HC (n=5), RRMS (n=8), BMS (n=12), and SPMS (n=33). Hierarchical clustering of soluble factors and patient groups was completed in R using divisive clustering methodology (DIANA) from the cluster package.

FIG. 2A-2C shows correlation analysis of plasma cytokine levels against age, disease duration, and platelet count. Ages of all participants (A) and disease duration (B) of all MS patients in this study were analyzed using the Kruskal-Wallis test. Statistically significant P values between groups calculated using Dunn's adjusted multiple comparison are shown above horizontal lines. Spearman rank correlation analysis was performed using the plasma levels of 8 most significantly different cytokines among different groups against age, disease duration, and circulating platelet count of corresponding individuals. Their correlation coefficient values are shown in scale of color as matrix (C). HC (n=5), RRMS (n=8), BMS (n=12), and SPMS (n=32).

FIG. 3A-3C show that plasma biomarkers discriminate SPMS from RRMS. (A) Column scatter graph of plasma concentrations of MCP1/CCL2 and sCD40L of RRMS and SPMS patients. P value using Mann-Whitney test are shown above the line. (B) ROC analysis using plasma concentrations of MCP1/CCL2 and sCD40L derived from RRMS and SPMS groups. (C) Multiple logistic regression analysis using plasma concentration of sCD40L and MCP1/CCL2 derived from patients of both RRMS and SPMS groups. RRMS (n=8), SPMS (n=30). ROC, receiver-operating characteristic; AUC, area under curve.

FIG. 4A-4B show that plasma levels of MCP1/CCL2 discriminate BMS from RRMS. Plasma concentration of MCP1/CCL2 derived from RRMS and BMS patients were compared along with their ROC plots. (A) Column scatter graph comparing plasma level of MCP1/CCL2 of RRMS group (n=8) with BMS group (n=12). P value using Mann-Whitney test are shown above the line. (B) ROC analysis using MCP/CCL2 to discriminate BMS from RRMS. ROC, receiver-operating characteristic; AUC, area under curve.

FIG. 5A-C show that plasma levels of sCD40L and IFNγ discriminate SPMS from BMS. Plasma concentration of sCD40L and IFNγ in BMS and SPMS patients were compared along with their ROC plots. (A) Column scatter graph of plasma concentrations of sCD40L and IFNγ derived from BMS and SPMS patients. P value using Mann-Whitney test are shown above the line. (B) ROC analysis using plasma concentrations of sCD40L and IFNγ derived from BMS and SPMS groups. (C) Multiple logistic regression analysis combining plasma concentration of sCD40L and IFNγ derived from patients of both BMS and SPMS groups. n: BMS=12, SPMS=32. ROC, receiver-operating characteristic; AUC, area under curve.

FIG. 6A-6B show that MCP1/CCL2 and sCD40L are significantly correlated with MS disease progression. Spearman correlation analysis was performed using plasma levels of MCP1/CCL2 (A) and sCD40L (B) against corresponding EDSS scores in all participants. r is the Spearman correlation coefficient. The solid line is the best fit line of linear regression with 95% CI (dotted line).

FIG. 7 shows a graphic summary of the results presented herein. RRMS (green) becomes SPMS (orange) over time with increased disability; BMS (blue) does not accumulate disability despite long duration of disease. Progressing to SPMS from RRMS (indicated with a half orange/half green arrow), sCD40L and MCP1 are increased. Comparing BMS to RRMS (indicated with a half blue/half green arrow), only MCP1 is increased. Although both BMS and SPMS have long disease duration, SPMS exhibits increased sCD40L and IFNγ compared to non-progressive BMS (indicated with a half orange/half blue arrow). Both MCP1 and sCD40L were found to be correlated with EDSS.

FIG. 8 shows neurofilament light chain (NFL) levels in control subjects (HC) and in subjects with BMS, RRMS, and SPMS. Results were analyzed using a Kruskal-Wallis test. NFL levels are significantly higher in subjects having SPMS compared to controls (p=0.0005) and significantly higher in subjects having SPMS compared to subjects having RRMS (p=0.0349). NFL levels are also significantly higher in subjects having BMS compared to controls (p=0.0486).

FIG. 9 shows that NFL levels correlate with sCD40L levels in MS.

FIG. 10 shows that NFL levels correlate with sCD40L levels to predict MS disease progression. Elevated levels of NFL and sCD40L are indicative of SPMS, whereas lower levels of NFL and sCD40L are indicative of BMS.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies, or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

The terms “multiple sclerosis” or “MS” are used interchangeably herein. Multiple sclerosis is an autoimmune disease associated with the central nervous system (CNS). The pathology of MS is characterized by T- and B-lymphocyte and innate immune cell infiltration into the CNS, myelin sheath breakdown, oligodendrocyte damage, and axonal degeneration. The term “multiple sclerosis” is inclusive of many times of multiple sclerosis, including non-progressive benign MS (NPMS, or BMS), relapsing-remitting MS (RRMS), and secondary progressive MS (SPMS).

The terms “non-progressive benign MS”, “NPMS”, and “BMS” are used interchangeably herein to refer to a rare type of multiple sclerosis characterized by mild relapses, and a lack of disease progression (e.g. lack of increasing disease severity) without the use of disease-modifying therapies. BMS occurs in about 5-10% of MS cases.

The terms “relapsing-remitting MS” or “RRMS” are used interchangeably herein to refer to a type of multiple sclerosis characterized by periods of relapse (e.g. noticeable disease symptoms) and remission (e.g. improvement of disease symptoms, or absence of disease symptoms). RRMS typically progresses to secondary progressive MS (SPMS). The terms “secondary progressive MS” or “SPMS” are used interchangeably herein to refer to a more severe form of multiple sclerosis characterized by less distinct periods of relapse and remission. SPMS is characterized by a general worsening of disease symptoms, more severe relapses, and/or more frequent relapses and in some instances, a worsening recovery from relapse.

As used herein, the terms “treat,” “treatment,” and “treating” refer to reducing the amount or severity of a particular condition, disease state (e.g., multiple sclerosis) or symptoms thereof, in a subject presently experiencing or afflicted with the condition or disease state. The terms do not necessarily indicate complete treatment (e.g., total elimination of the condition, disease, or symptoms thereof). “Treatment,” encompasses any administration or application of a therapeutic or technique for a disease (e.g., in a mammal, including a human), and includes inhibiting the disease, arresting its development, relieving the disease, causing regression, or restoring or repairing a lost, missing, or defective function; or stimulating an inefficient process. In some embodiments, “treating multiple sclerosis” refers to increasing the amount of time between relapses, improving recovery from relapses, decreasing the severity of relapses, and/or other similar improvements in MS symptoms.

As used herein, the terms “prevent,” “prevention,” and preventing” refer to reducing the likelihood of a particular condition or disease state (e.g., multiple sclerosis) from occurring in a subject not presently experiencing or afflicted with the condition or disease state. The terms do not necessarily indicate complete or absolute prevention. In some embodiments “preventing multiple sclerosis” may refer to reducing the likelihood of RRMS from progressing to SPMS.

### DETAILED DESCRIPTION

Previous studies examining MS-associated factors have focused primarily on RRMS, and those that studied SPMS usually compared them to RRMS. However, individuals with RRMS and SPMS typically differ on a variety of features including age, disease duration, disease trajectory, and the use of DMTs. Thus, differentially expressed factors may be unrelated to disease progression. Aging affects the levels of many cytokines and influences the levels of soluble factors previously associated with disease progression, including neurofilament light. While cerebrospinal fluid (CSF) derived factors typically are closely tied to disease pathology for most CNS diseases, MS involves a dysregulation of immunity in both central and peripheral compartments.

Herein, plasma levels of cytokines, chemokines, and growth factors were analyzed in different forms of MS, including RRMS, SPMS, and BMS. A novel approach was taken by using non-progressive BMS, age- and disease duration-matched to progressive SPMS, to dissect progression-specific mechanisms. A set of plasma biomarkers was identified that differentiates between various forms of MS, which are especially useful for progressive MS and leads to appropriate selection of therapeutic interventions. Specifically, herein it was found that plasma levels of soluble CD40L (sCD40L), Monocyte Chemoattractant Protein-1 (MCP-1, also referred to herein as “MCP1” or “MCP1/CCL2”), neurofilament light chain (NFL), and gamma interferon (IFNγ) find use to determine different disease course of multiple sclerosis (MS).

In some embodiments, combined elevation of plasma levels of sCD40L and MCP-1 is used to identify those patients whose disease are progressed from relapsing-remitting MS (RRMS) to secondary progressive MS (SPMS). In some embodiments, plasma level of MCP-1 differentiates those who have non-progressive (BMS) from those with RRMS. In some embodiments, progressive SPMS is prognosed from non-progressive BMS by increased levels of sCD40L and IFNγ.

In some aspects, provided herein are methods. In some embodiments, provided herein is a method comprising measuring levels of soluble CD40L (sCD40L), Monocyte Chemoattractant Protein-1 (MCP-1) and gamma interferon (IFNγ) in a sample obtained from a subject having or suspected of having multiple sclerosis (MS). In some embodiments, provided herein is a method comprising measuring levels of soluble Monocyte Chemoattractant Protein-1 (MCP-1), gamma interferon (IFNγ), and neurofilament light chain (NFL) in a sample obtained from a subject having or suspected of having multiple sclerosis (MS).

The sample may be any suitable sample type obtained from the subject. In some embodiments, the sample is a blood sample, a serum sample, or a plasma sample. In some embodiments, the sample is a plasma sample. In some embodiments, the levels of soluble CD40L (sCD40L), Monocyte Chemoattractant Protein-1 (MCP-1) and gamma interferon (IFNγ) are measured by an immunoassay. In some embodiments, levels of soluble Monocyte Chemoattractant Protein-1 (MCP-1), gamma interferon (IFNγ), and neurofilament light chain (NFL) are measured by an immunoassay.

Any suitable immunoassay may be used, including sandwich immunoassays (sandwich immunoassay (e.g., monoclonal-monoclonal sandwich immunoassays, monoclonal-polyclonal sandwich immunoassays), radioimmunoassay (RIA), a counting immunoassay (CIA), an enzyme immunoassays (EIA) or an enzyme-linked immunosorbent assays (ELISA), a fluoroimmnoassay (FIA), a chemiluminescence immunoassay (CLIA). In some embodiments, the immunoassay is a bead-based immunoassay.

In some embodiments, the methods further comprise determining whether the level of MCP-1 in the sample is elevated compared to a cutoff value. In some embodiments, the methods further comprise determining whether the level of MCP-1 in the sample is elevated compared to a cutoff value of at least about 350 μg/mL to about 380 μg/mL. In some embodiments, the methods comprise determining whether the level of MCP-1 in the sample is elevated compared to a cutoff value of about 350 μg/mL, about 355 μg/mL, about 360 μg/mL, about 365 μg/mL, about 370 μg/mL, about 375 μg/mL, or about 380 μg/mL. In some embodiments, the cutoff value is 369.4 pg/mL. In some embodiments, the cutoff value is 371.9 pg/mL.

In some embodiments, the methods further comprise determining whether the level of sCD40L in the sample is elevated compared to a cutoff value. In some embodiments, the methods comprise determining whether the level of sCD40L in the sample is elevated compared to a cutoff value of at least about 450 μg/mL. For example, in some embodiments the methods comprise determining whether the level of sCD40L in the sample is elevated compared to a cutoff value of about 450 μg/mL, about 455 μg/mL, about 460 μg/mL, about 465 μg/mL, about 470 μg/mL, about 475 μg/mL, about 480 μg/mL, about 485 μg/mL, about 490 μg/mL, about 495 μg/mL, about 500 μg/mL, about 600 μg/mL, about 700 μg/mL, about 800 μg/mL, about 900 μg/mL, about 1000 μg/mL, about 1200 μg/mL, about 1400 μg/mL, about 1600 μg/mL, about 1800 μg/mL, about 2000 μg/mL, about 2100, about 2200, or about 2300 μg/mL. In some embodiments, the cutoff value is 487.6 pg/mL. In some embodiments, the cutoff value is 2206 μg/mL.

In some embodiments, the methods further comprise determining whether the level of IFNγ in the sample is elevated compared to a cutoff value. In some embodiments, the methods comprise determining whether the level of IFNγ in the sample is elevated compared to a cutoff value of about 1 μg/mL to about 10 μg/mL. For example, in some embodiments the cutoff value is about 1 μg/mL, about 2 μg/mL, about 3 μg/mL, about 4 μg/mL, about 5 μg/mL, about 6 μg/mL, about 7 μg/mL, about 8 μg/mL, about 9 μg/mL, or about 10 μg/mL. In some embodiments, the cutoff value is 7.805 pg/mL.

In some embodiments, the methods further comprise determining whether the level of NFL in the sample is elevated compared to a cutoff value. In some embodiments, the methods comprise determining whether the level of IFNγ in the sample is elevated compared to a cutoff value of about 6 to about 12 μg/mL. For example, in some embodiments the cutoff value is about 6 μg/mL, about 7 μg/mL, about 8 μg/mL, about 9 μg/mL, about 10 μg/mL, about 11 μg/mL, or about 10 μg/mL.

In some aspects, provided herein are methods of stratifying multiple sclerosis in a subject. For example, in some embodiments the methods described herein are useful for identifying subjects as having benign non-progressive multiple sclerosis (BMS), relapsing-remitting multiple sclerosis (RRMS), or secondary progressive multiple sclerosis (SPMS). For example, in some embodiments the methods comprise measuring levels of one or more biomarkers selected from soluble CD40L (sCD40L), Monocyte Chemoattractant Protein-1 (MCP-1) and/or gamma interferon (IFNγ) in a sample obtained from a subject and stratifying the subject into a multiple sclerosis type (e.g. BMS, RRMS, or SPMS) based upon the levels of the one or more biomarkers measured in the sample. As another example, in some embodiments the methods comprise measuring levels of one or more biomarkers selected from soluble CD40L (sCD40L), neurofilament light chain (NFL), Monocyte Chemoattractant Protein-1 (MCP-1) and/or gamma interferon (IFNγ) in a sample obtained from a subject and stratifying the subject into a multiple sclerosis type (e.g. BMS, RRMS, or SPMS) based upon the levels of the one or more biomarkers measured in the sample. In some embodiments, the methods stratifying the subject into a multiple sclerosis type based upon whether the levels of the one or more biomarkers in the sample are elevated compared to a cutoff level for the respective biomarker.

In some embodiments, provided herein is a method of stratifying multiple sclerosis in a subject, comprising measuring levels of soluble CD40L (sCD40L), Monocyte Chemoattractant Protein-1 (MCP-1) and gamma interferon (IFNγ) in a sample obtained from a subject having or suspected of having multiple sclerosis. In some embodiments, the method comprises identifying the subject as having relapsing-remitting multiple sclerosis when the level of MCP-1 in the sample is not elevated compared to a cutoff value. In some embodiments, the method comprises identifying the subject as having progressed from relapsing-remitting multiple sclerosis to secondary progressive multiple sclerosis when the level of sCD40L in the sample is elevated compared to a cutoff value and the level of MCP-1 in the sample is elevated compared to a cutoff value. In some embodiments, the method comprises identifying the subject as having progressed from relapsing-remitting multiple sclerosis to secondary progressive multiple sclerosis when the level of sCD40L in the sample is elevated compared to a cutoff value and the level of IFNγ in the sample is elevated compared to a cutoff value. In some embodiments, the method comprises identifying the subject as having non-progressive benign multiple sclerosis when the level of MCP-1 in the sample is elevated compared to a cutoff value and the level of sCD40L in the sample is not elevated compared to a cutoff value. In some embodiments, the method comprises identifying the subject as having non-progressive benign multiple sclerosis when the level of MCP-1 in the sample is elevated compared to a cutoff value and the level of IFNγ in the sample is not elevated compared to a cutoff value. In some embodiments, the sample is a plasma sample.

In some embodiments, provided herein is a method of stratifying multiple sclerosis in a subject, comprising measuring levels of soluble CD40L (sCD40L), Monocyte Chemoattractant Protein-1 (MCP-1) and/or gamma interferon (IFNγ) in a plasma sample obtained from a subject having or suspected of having multiple sclerosis. In some embodiments, the method further comprises identifying the subject as having relapsing-remitting multiple sclerosis when the level of MCP-1 in the sample is less than 369.4 pg/mL. In some embodiments, the method further comprises identifying the subject as having progressed from relapsing-remitting multiple sclerosis to secondary progressive multiple sclerosis when the level of sCD40L in the sample is greater than 487.6 pg/mL and the level of MCP-1 in the sample is greater than 369.5 pg/mL. In some embodiments, the method further comprises identifying the subject as having progressed from relapsing-remitting multiple sclerosis to secondary progressive multiple sclerosis when the level of sCD40L in the sample is greater than 2206 μg/mL and the level of IFNγ in the sample is greater than 7.805 pg/mL. In some embodiments, the method further comprises identifying the subject as having non-progressive benign multiple sclerosis when the level of MCP-1 in the sample is greater than 371.9 pg/mL and the level of sCD40L is less than 2206 μg/mL. In some embodiments, the method further comprises identifying the subject as having non-progressive benign multiple sclerosis when the level of MCP-1 in the sample is greater than 371.9 pg/mL and the level of IFNγ in the sample is less than 7.806 pg/mL.

In some embodiments, provided herein is a method comprising measuring levels of soluble CD40L (sCD40L) and Monocyte Chemoattractant Protein-1 (MCP-1) in a sample obtained from a subject having or suspected of having multiple sclerosis, and identifying the subject as having relapsing-remitting multiple sclerosis or secondary progressive multiple sclerosis based upon the levels measured in the sample. In some embodiments, the method comprises identifying the subject as having relapsing-remitting multiple sclerosis when the level of MCP-1 in the sample is not elevated compared to a cutoff value. In some embodiments, the method comprises identifying the subject as having progressed from relapsing-remitting multiple sclerosis to secondary progressive multiple sclerosis when the level of MCP-1 in the sample is elevated compared to a cutoff value and the level of sCD40L in the sample is elevated compared to a cutoff value. In some embodiments, the sample is a plasma sample. In some embodiments, the cutoff value for MCP-1 is 369.4 pg/mL. In some embodiments, the cutoff value for sCD40L is 487.6 pg/mL. In some embodiments, the cutoff value for MCP-1 is 369.4 pg/mL and the cutoff value for sCD40L is 487.6 pg/mL.

In some embodiments, provided herein is a method comprising measuring a level of Monocyte Chemoattractant Protein-1 (MCP-1) in a sample obtained from a subject having or suspected of having multiple sclerosis, and identifying the subject as having RRMS or BMS based upon whether the level of MCP-1 in the sample is elevated compared to a cutoff value. In some embodiments, the subject is identified as having relapsing-remitting multiple sclerosis when the level of MCP-1 in the sample is not elevated compared to a cutoff value. In some embodiments, the subject is identified as having benign non-progressive benign multiple sclerosis when the level of MCP-1 in the sample is elevated compared to a cutoff value. In some embodiments, the method further comprises measuring a level of sCD40L and/or a level of IFNγ in the sample. In some embodiments, the subject is identified as having benign non-progressive benign multiple sclerosis when the level of MCP-1 in the sample is elevated compared to a cutoff value and the level of sCD40L is not elevated in the sample compared to a cutoff value. In some embodiments, the subject is identified as having benign non-progressive benign multiple sclerosis when the level of MCP-1 in the sample is elevated compared to a cutoff value and the level of IFNγ in the sample is not elevated compared to a cutoff value. In some embodiments, the cutoff value for MCP-1 is 371.9 pg/mL. In some embodiments, the cutoff value for sCD40L is 2206 μg/mL and the cutoff value for IFNγ is 7.805.

In some embodiments, provided herein is a method comprising measuring levels of soluble CD40L (sCD40L) and gamma interferon (IFNγ) in a sample obtained from a subject having or suspected of having multiple sclerosis, and identifying the subject as having progressed from relapsing-remitting multiple sclerosis to secondary progressive multiple sclerosis based upon the levels in the sample. In some embodiments, the subject is identified as having progressed from relapsing-remitting multiple sclerosis to secondary progressive multiple sclerosis when the level of sCD40L in the sample is elevated compared to a cutoff value and the level of IFNγ in the sample is elevated compared to a cutoff value. In some embodiments, the sample is a plasma sample. In some embodiments, the cutoff value for sCD40L is 2206 μg/mL and the cutoff value for IFNγ is 7.805 pg/mL.

In some embodiments, provided herein are methods of stratifying multiple sclerosis in a subject. In some embodiments, provided herein is a method of stratifying multiple sclerosis in a subject, comprising measuring levels of Monocyte Chemoattractant Protein-1 (MCP-1), gamma interferon (IFNγ), and at least one of soluble CD40L (sCD40L) or neurofilament light chain (NFL) in a sample obtained from a subject having or suspected of having multiple sclerosis. In some embodiments, the method comprises identifying the subject as having relapsing-remitting multiple sclerosis when the level of MCP-1 in the sample is not elevated compared to a cutoff value. In some embodiments, the method comprises identifying the subject as having progressed from relapsing-remitting multiple sclerosis to secondary progressive multiple sclerosis when the level of NFL or the level of sCD40L in the sample is elevated compared to a cutoff value and the level of MCP-1 in the sample is elevated compared to a cutoff value, or the level of IFNγ in the sample is elevated compared to a cutoff value. In some embodiments, the method comprises identifying the subject as having non-progressive benign multiple sclerosis when the level of MCP-1 in the sample is elevated compared to a cutoff value and (i) the level of sCD40L in the sample is not elevated compared to a cutoff value or (ii) the level of IFNγ in the sample is not elevated compared to a cutoff value. For example, in some embodiments the method comprises identifying the subject as having non-progressive benign multiple sclerosis when the level of MCP-1 in the sample is elevated compared to a cutoff value and the level of sCD40L in the sample is not elevated compared to a cutoff value. As another example, in some embodiments the method comprises identifying the subject as having non-progressive benign multiple sclerosis when the level of MCP-1 in the sample is elevated compared to a cutoff value and the level of IFNγ in the sample is not elevated compared to a cutoff value.

In some embodiments, provided herein is a method of stratifying multiple sclerosis in a subject, comprising measuring levels of soluble CD40L (sCD40L), neurofilament light chain (NFL), Monocyte Chemoattractant Protein-1 (MCP-1) and/or gamma interferon (IFNγ) in a plasma sample obtained from a subject having or suspected of having multiple sclerosis. In some embodiments, the method comprises identifying the subject as having relapsing-remitting multiple sclerosis when the level of MCP-1 in the sample is less than 369.4 pg/mL. In some embodiments, the method comprises identifying the subject as having progressed from relapsing-remitting multiple sclerosis to secondary progressive multiple sclerosis when: (i) the level of sCD40L in the sample is greater than 487.6 pg/mL and the level of MCP-1 in the sample is greater than 369.5 pg/mL, (ii) the level of sCD40L in the sample is greater than 2206 μg/mL and the level of IFNγ in the sample is greater than 7.805 pg/mL, (iii) the level of NFL in the sample is greater than 10 μg/mL, (iv) the level of NFL in the sample is greater than 10 μg/mL and the level of MCP-1 in the sample is greater than 369.5 pg/mL, or (v) the level of NFL in the sample is greater than 10 μg/mL and the level of IFNγ in the sample is greater than 7.805 pg/mL. In some embodiments the method comprises identifying the subject as having non-progressive benign multiple sclerosis when (i) the level of MCP-1 in the sample is greater than 371.9 pg/mL and the level of sCD40L is less than 2206 μg/mL, (ii) the level of MCP-1 in the sample is greater than 371.9 pg/mL and the level of IFNγ in the sample is less than 7.806 pg/mL, or (iii) the level of MCP-1 in the sample is greater than 371.9 pg/mL and the level of NFL in the sample is less than 10 μg/mL.

In some embodiments, provided herein is a method comprising measuring levels of Monocyte Chemoattractant Protein-1 (MCP-1) and at least one of soluble CD40L (sCD40L) or neurofilament light chain (NFL) in a sample obtained from a subject having or suspected of having multiple sclerosis. In some embodiments, the method comprises identifying the subject as having relapsing-remitting multiple sclerosis when the level of MCP-1 in the sample is not elevated compared to a cutoff value. In some embodiments, the method comprises identifying the subject as having progressed from relapsing-remitting multiple sclerosis to secondary progressive multiple sclerosis when the level of MCP-1 in the sample is elevated compared to a cutoff value and the level of at least one of sCD40L or NFL in the sample is elevated compared to a cutoff value. In some embodiments, the cutoff value for MCP-1 is 369.4 pg/mL. In some embodiments, the cutoff value for sCD40L is 487.6 pg/mL or the cutoff value for NFL is 10 μg/mL.

In some embodiments, provided herein is a method comprising measuring levels of neurofilament light chain (NFL) and at least one of soluble CD40L (sCD40L) or gamma interferon (IFNγ) in a sample obtained from a subject having or suspected of having multiple sclerosis. In some embodiments, the method comprises identifying the subject as having non-progressive benign multiple sclerosis when the level of NFL in the sample is increased compared to a cutoff value and the level of at least one of sCD40L or IFNγ is not increased compared to a cutoff value. In some embodiments, the method comprises identifying the subject as having progressed from relapsing-remitting multiple sclerosis to secondary progressive multiple sclerosis when the level of NFL in the sample is elevated compared to a cutoff value and the level of at least one of sCD40L or IFNγ) in the sample is elevated compared to a cutoff value. In some embodiments, the cutoff value for NFL is 6 pg/mL and (i) the cutoff value for sCD40L is 2206 μg/mL or (ii) the cutoff value for IFNγ is 7.805 pg/mL.

In some embodiments, provided herein is a method comprising measuring a level of neurofilament light chain (NFL) in a sample obtained from a subject having or suspected of having multiple sclerosis, and identifying the subject as having secondary progressive multiple sclerosis when the level of neurofilament light chain (NFL) in the sample is elevated compared to a cutoff value. In some embodiments, the cutoff value is 10 μg/mL.

For any of the embodiments described herein, the cutoff value for MCP-1 may be at least about 350 μg/mL to about 380 μg/mL. For example, in some embodiments the cutoff value for MCP-1 is about 350 μg/mL, about 355 μg/mL, about 360 μg/mL, about 365 μg/mL, about 370 pg/mL, about 375 μg/mL, or about 380 μg/mL. In some embodiments, the cutoff value is 369.4 pg/mL. In some embodiments, the cutoff value is 371.9 pg/mL.

For any of the embodiments described herein, the cutoff value for sCD40L may be at least about 450 μg/mL. For example, in some embodiments, the cutoff value for sCD40L is about 450 μg/mL, about 455 μg/mL, about 460 μg/mL, about 465 μg/mL, about 470 μg/mL, about 475 μg/mL, about 480 μg/mL, about 485 μg/mL, about 490 μg/mL, about 495 μg/mL, about 500 μg/mL, about 600 μg/mL, about 700 μg/mL, about 800 μg/mL, about 900 μg/mL, about 1000 μg/mL, about 1200 μg/mL, about 1400 μg/mL, about 1600 μg/mL, about 1800 μg/mL, about 2000 μg/mL, about 2100, about 2200, or about 2300 μg/mL. In some embodiments, the cutoff value is 487.6 pg/mL. In some embodiments, the cutoff value is 2206 μg/mL.

For any of the embodiments described herein, the cutoff value for IFNγ may be about 1 μg/mL to about 10 μg/mL. For example, in some embodiments the cutoff value for IFNγ is about 1 μg/mL, about 2 μg/mL, about 3 μg/mL, about 4 μg/mL, about 5 μg/mL, about 6 μg/mL, about 7 μg/mL, about 8 μg/mL, about 9 μg/mL, or about 10 μg/mL. In some embodiments, the cutoff value is 7.805 pg/mL.

For any of the embodiments described herein, the sample may be any suitable sample obtained from the subject, including but not limited to a blood sample, a serum sample, or a plasma sample. In some embodiments, the sample is a plasma sample.

For any of the embodiments described herein, the methods may comprise treating the subject with an appropriate multiple sclerosis therapy based upon whether the subject is identified as having BMS, RRMS, or SPMS. In some embodiments, the methods comprise monitoring the subject identified as having BMS. In some embodiments, the methods comprise treating the subject identified as having relapsing-remitting multiple sclerosis with a disease-modifying therapy (DMT). In some embodiments, the DMT comprises one or more therapies selected from beta interferon, glatiramer acetate, fingolimod, dimethyl fumarate, diroximel fumarate, teriflunomide, Siponimod, cladribine, a monoclonal antibody, or mitoxantrone. In some embodiments, the monoclonal antibody is selected from, ocrelizumab, natalizumab, and alemtuzumab. In some embodiments, the method comprises treating the subject identified as having secondary progressive multiple sclerosis with one or more therapies selected from stem cell therapy, steroids, beta interferon, cladribine, and Siponimod. In some embodiments, the method further comprises providing to the subject a therapy that is not a DMT described above, but an alternative therapy to relieve one or more symptoms of MS. Suitable alternative therapies include, for example, corticosteroids, plasma exchange, physical therapy, muscle relaxants, medications to reduce fatigue (e.g. amantadine, modafinil, methylphenidate, anti-depressants (e.g. SSRIs), medications to increase walking speed (dalmampridine), medications for pain management, medications to treat sexual dysfunction, insomnia, or bladder and bowel control problems, and the like.

In some aspects, provided herein are kits. In some embodiments, provided herein are kits for measuring one or more biomarkers selected from soluble CD40L (sCD40L), Monocyte Chemoattractant Protein-1 (MCP-1) and/or gamma interferon (IFNγ) in a sample. In some embodiments, the kits comprise reagents for detecting the one or more biomarkers by an immunoassay (e.g. buffers, antibodies, plates, etc.). In some embodiments, the kit further comprises instructions for use. In some embodiments, the kit further comprises materials for collection and/or storage of a sample, such as for collection and/or storage of a plasma sample from a subject. For example, the kit may further comprise syringes, tubes, preservatives, and the like.

### EXAMPLES

### Example 1

### Materials and Methods

Study Approval and Participating Subjects Recruitment: The study consisted of a total of 52 MS participants (32 SPMS, 8 RRMS, and 12 BMS) and 5 healthy control (HC) participants. HC and MS participants were recruited from the University of Michigan Multiple Sclerosis Clinic and the Autoimmunity Center of Excellence. RRMS and SPMS were defined by the 2010 Revised McDonald criteria and were not treated with disease-modifying therapy (DMT) at the time of the study. Non-progressive BMS patients were not treated with disease modifying therapy and were defined as having an Expanded Disability Status Scale (EDSS) score≤3 with more than 15 years of disease duration. The SPMS group included baseline patients in the AMS04 study prior to randomization. The AMS04 is a multicentered SPMS mechanistic study of siponimod. Detailed inclusion and exclusion criteria can be found in clinicaltrials.gov identifier NCT02330965. SPMS patients had a unified and documented recent progression as defined by a progressive increase in disability (of at least 6 months duration) in the absence of relapses or independent of relapses: EDSS progression in the 2 years prior to the study of ≥1 point for patients with EDSS<6.0 at baseline, and ≥0.5 point for patients with EDSS≥6.0 at baseline. Written informed consent was obtained from all patients prior to participation in this study, which was approved by the University of Michigan Institutional Review Board. Clinical assessments including EDSS are performed on all patients. The demographic and disease-associated characteristics of the participants are shown in Table 1.

TABLE 1
Demographics of Participants of this Study.

| | | | | | MS | |
|---|---|---|---|---|---|---|
| | NUMBER | | | RACE | DIAGNOSIS | |
| | OF | AGE | FEMALE/ | (WHITE/ | DURATION | EDSS |
| GROUPS | PATIENTS | (SD) | MALE | OTHER) | (SD) | (SD) |
| HC | 5 | 56.3 | 4/1 | 5/0 | — | — |
| | | (7.8) | | | | |
| RRMS | 8 | 40.7 | 7/1 | 7/1 | 3.2 | 1.2 |
| | | (13.5) | | | (3.0) | (.9) |
| BMS | 12 | 57.0 | 11/1 | 12/0 | 24.9 | 1.2 |
| | | (7.3) | | | (9.8) | (.7) |
| SPMS | 32 | 53.0 | 24/8 | 29/3 | 20.4 | 5.7 |
| | | (7.1) | | | (10.5) | (1.3) |

Age, average age of the group; BMS, non-progressive benign MS; EDSS, The Expanded Disability Status Scale; HC, healthy control; MS diagnosis duration, average MS disease duration after MS diagnosis; RRMS, relapsing-remitting MS; SD, standard deviation; SPMS, secondary progressive MS.

Peripheral Blood Mononuclear Cells (PBMC) and Plasma Isolation: About 60 mL of heparinized peripheral blood was collected from each participant in Sodium

Heparin Vacutainer™ (BD Biosciences). Plasma was collected from a clear top layer after a spin of 400 g for 10 min, followed by centrifugation at 1800 g for 15 min without a break. The clear plasma was aliquoted and stored at 80° C. before use.

Luminex Assay: The frozen plasma collected from HC and participants with RRMS, BMS, and SPMS was used to measure chemokine and cytokine concentrations. Luminex assays of the plasma cytokine profile were measured using the HCYTMAG-60K-PX33 kit purchased from Sigma-Millipore (Burlington, MA, USA), according to the manufacturer's recommended protocol. The kit is designed to detect these inflammation-related cytokines and chemokines: EGF, FGF-2, Eotaxin, G-CSF, Flt3L, GM-CSF, Fractalkine (CX3CL1), IFN-α2, IFNγ, Groα (CXCL1), IL-10, IL-12p40 MDC (CCL22), IL-12p70, IL-13, IL-15, sCD40L, IL-17A, IL-9, IL-2, IL-3, IL-4, IL-5, IL-6, IL-7, IL-8 (CXCL8), IP-10 (CXCL10), MCP-1 (CCL2), MIP-1α (CCL3), MIP-1β (CCL4), TNFα, TNFβ (LTα), and VEGF. All plasma samples were spun for 10 s at 16000 g, and the clear supernatants were added to the wells of a Luminex assay plate. Each sample was assayed in duplicate.

Statistical Analysis: The plasma derived from HC, RRMS, BMS, and SPMS were analyzed using the Luminex kit from Millipore. Kruskal-Wallis test and multiple comparison with Dunn's modification between different groups were performed. Median of plasma concentration (pg/mL) are presented with 25-75% range. Nonparametric Mann-Whitney U tests were used when data derived from 2 groups were analyzed. Receiver operator characteristic (ROC) analysis, multiple logistic regression analysis and Spearman correlation coefficient, and 95% CI and two-tail P values were calculated using Prism GraphPad software (version 8.4.3). P<0.05 and r>0.3 for correlation are considered statistically significant. Z-scores used in the heatmap were calculated according to the formula: z-score is z=(xμ)/Υ, where x is the individual raw soluble factor concentration, μ is the population mean of the soluble factor of all sample analyzed, and σ is the population standard deviation.

### Results

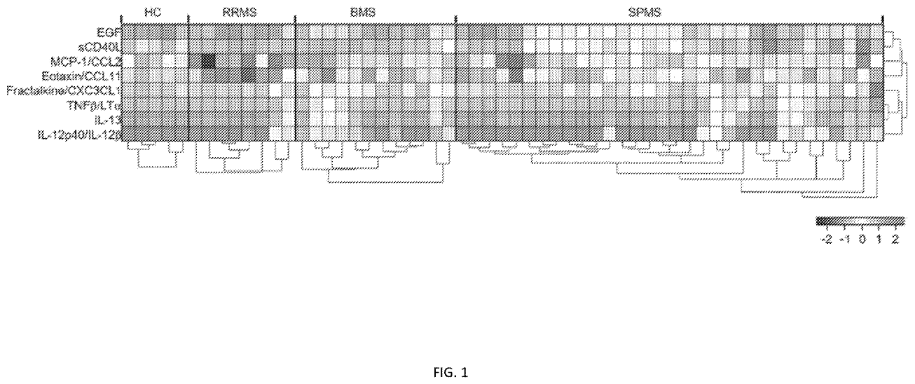

Overview: To identify cytokine, chemokine, or soluble factor in plasma that differentiate between HC and MS participants, or among different MS types, plasma concentration of 33 cytokine/chemokines were measured with Luminex using plasma samples derived from 4 groups of individuals including HC, RRMS, BMS, and SPMS. The resulting concentrations were analyzed using the Kruskal-Wallis method comparing all 4 groups. Differences in EGF (P≤0.0001), soluble CD40L (sCD40L) (P=0.0009), MCP-1/CCL2 (P=0.0029), Fractalkine/CX3CL1 (P=0.0128), IL-13 (P=0.0358), Eotaxin (P=0.0360), IL-12 (p40) (P=0.0420), and TNFβ/LTα (P=0.0482) (FIG. 1, Table 2) all showed statistical significance (P<0.05). IL-8/CXCL8 (P=0.0748), IFNγ (P=0.0812), IL-4 (P=0.0848), and IL-15 (P=0.0905) showed a trend toward significance (Table 3). No statistically significant differences were detected for the following cytokines and chemokines: MIP-1a/CCL3, IL-10, IL-6, VEGF, IFN-α2, TNFβ, IL-7, MDC/CCL22, IP-10/CXCL10, IL-9, IL-17A, FGF2, GM-CSF, G-CSF, MIP-1β/CCL4, IL-5, IL-2, IL-3, GROα/CXCL1, IL-12 (p70), and Flt-3L (Table 3). Several circulating cytokines/chemokines were found to be significantly different between the different types of MS (FIG. 1, Table 2). Using Dunn's multiple comparison, significant differences between HC and SPMS were found in circulating EGF (P=0.007) and Fractalkine/CX3CL1 (P=0.046) (Table 2). Several cytokines were found to be significantly different comparing RRMS with SPMS, including EGF (P=0.0016), MCP1/CCL2 (P=0.0016), sCD40L (P=0.0053), and Eotaxin (P=. 0427). No soluble biomarkers were significantly different between RRMS and BMS, while TNFβ/LTα (P=0.0583), IL-12p40/IL-12B (P=0.0616), and IL-13 (P=0.0776) showed a trend of difference. sCD40L was the only soluble factor that was found to be significantly different between BMS and SPMS (P=0.014) (Table 2).

TABLE 2
Significantly Different Plasma Cytokine/Chemokine/Growth Factor Levels
Among Different Types of Multiple Sclerosis and Healthy Controls.

| | | | | | | DUNN'S MULTIPLE x | | | | | |
|---|---|---|---|---|---|---|---|---|---|---|---|
| | KRUSKAL- | | | | | COMPARISON (P VALUE) | | | | | |
| | WALLIS TEST | MEDIAN (25%-75% RANGE) | | | | HC VS | | | RRMS VS | | BMS |
| ANALYTE | P VALUE | HC | RRMS | BMS | SPMS | RRMS | BMS | SPMS | BMS | SPMS | SPMS |
| EGF | <.0001 | 18.30 | 20.63 | 44.10 | 113.5 | >.9999 | .8213 | .007 | .8429 | .0016 | .1371 |
| | | (6.965- | (6.528- | (17.78- | (80.51- | | | | | | |
| | | 29.50) | 43.17) | 73.24) | 209.7) | | | | | | |
| sCD40L | .0009 | 925.4 | 324.32 | 384.6 | 2416 | >.9999 | >.9999 | >.9999 | >.9999 | .0053 | .014 |
| | | (471.1- | (199.4- | (125.2- | (587.4- | | | | | | |
| | | 1468) | 421.8) | 586.14) | 5107) | | | | | | |
| MCP1/CCL2 | .0029 | 501.5 | 311.4 | 440.3 | 554.7 | .3841 | >.9999 | >.9999 | .2976 | .0016 | .6333 |
| | | (392.1- | (263.1- | (359.1- | (442.5- | | | | | | |
| | | 583.1) | 359.8) | 598.8) | 700.3) | | | | | | |
| Fractalkine/ | .0128 | 34.62 | 65.85 | 183.2 | 147.4 | >.9999 | .0666 | .046 | .3523 | .2689 | >.9999 |
| CX3CL1 | | (18.31- | (39.40- | (80.28- | (72.97- | | | | | | |
| | | 84.08) | 105.0) | 337.9) | 263.3) | | | | | | |
| IL-13 | .0358 | .525 | .13 | 54.98 | 15.75 | >.9999 | .4602 | .841 | .0776 | .1354 | >.9999 |
| | | (.525- | (.13- | (.525- | (.525- | | | | | | |
| | | 4.075) | 47.66) | 253.7) | 168.0) | | | | | | |
| Eotaxin | .0360 | 476.8 | 201.9 | 423.5 | 598.8 | .1676 | >.9999 | >.9999 | .9355 | .0427 | >.9999 |
| | | (394.7- | (165.6- | (291.6- | (334.9- | | | | | | |
| | | 1067) | 481.6) | 778.9) | 888.2) | | | | | | |
| IL-12p40/ | .0420 | .95 | .3550 | 36.22 | .95 | >.9999 | .3291 | >.9999 | .0616 | .3292 | >.9999 |
| IL12B | | (.95- | (.19- | (4.76- | (.95- | | | | | | |
| | | 2.14) | 60.21) | 152.70) | 87.14) | | | | | | |
| TNFβ/LTα | .0482 | 475 | .115 | 54.23 | 8.70 | >.9999 | >.9999 | >.9999 | .0583 | .1182 | >.9999 |
| | | (.475- | (.115- | (.475- | (.475- | | | | | | |
| | | 18.08) | 14.10) | 400.9) | 298.5) | | | | | | |

Plasma derived from HC (n = 5), patients with RRMS (n = 8), BMS (n = 12), and SPMS (n = 32) were analyzed using Luminex kit from Millipore. Kruskal-Wallis test and multiple comparison with Dunn's modification between different groups were performed. Median of plasma concentration (pg/mL) is presented followed by 25-75% range in the brackets.

TABLE 3
Plasma cytokine concentration did not show significant difference among
different types of multiple sclerosis compared with healthy control

| | Kruskal- | | | | | | | | | | |
|---|---|---|---|---|---|---|---|---|---|---|---|
| | Wallis | | | | | | Dunn's Multiplex Comparison (P value) | | | | |
| | test | Median (25%-75% range) | | | | | HC vs | | RRMS vs | | BMS |
| Analytes | P value | HC | RRMS | BMS | SPMS | RRMS | BMS | SPMS | BMS | SPMS | SPMS |
| IL-8/ | 0.0748 | 5.82 | 11.75 | 14.22 | 43.47 | >0.9999 | 0.4122 | 0.119 | >0.9999 | 0.7161 | >0.9999 |
| CXCL8 | | (2.14- | (2.78- | (8.69- | (6.85- | | | | | | |
| | | 18.90) | 26.79) | 40.44) | 140.00) | | | | | | |
| IFNγ | 0.0812 | 6.55 | 5.7 | 16.61 | 6.55 | >0.9999 | 0.7176 | >0.9999 | 0.1656 | >0.9999 | 0.1344 |
| | | (4.54- | (2.32- | (9.195- | (2.678- | | | | | | |
| | | 12.32) | 24.10) | 64.32) | 17.24) | | | | | | |
| IL-4 | 0.0848 | 1.6 | 5.17 | 48.73 | 12.28 | >0.9999 | 0.2718 | >0.9999 | 0.1671 | >0.9999 | >0.8869 |
| | | (1.6- | (0.58- | (12.48- | (1.6- | | | | | | |
| | | 25.13) | 30.73) | 228.30) | 249.00) | | | | | | |
| IL-15 | 0.0905 | 1.42 | 1.935 | 12.46 | 3.96 | >0.9999 | 0.1059 | 0.43 | 0.7072 | >0.9999 | >0.9999 |
| | | (0.42- | (0.750- | (2.83- | (0.42- | | | | | | |
| | | 2.20) | 18.39) | 33.60) | 35.53) | | | | | | |
| MIP-1α/ | 0.1082 | 2.47 | 7.34 | 15.38 | 13.59 | >0.9999 | 0.3139 | 0.1963 | >0.9999 | >0.9999 | >0.9999 |
| CCL3 | | (1.12- | (0.48- | (4.94- | (4.07- | | | | | | |
| | | 6.09) | 15.66) | 22.68) | 27.44) | | | | | | |
| VEGF | 0.1429 | 78.34 | 34.64 | 98.26 | 85.08 | >0.9999 | >0.9999 | >0.9999 | 0.3293 | 0.1445 | >0.9999 |
| | | (43.20- | (4.49- | (48.14- | (60.55- | | | | | | |
| | | 114.50) | 85.82) | 123.70) | 133.10) | | | | | | |
| IL-6 | 0.1492 | 2.87 | 1.65 | 35.01 | 8.16 | >0.9999 | 0.5398 | >0.9999 | 0.278 | 0.9537 | >0.9999 |
| | | (0.39- | (0.35- | (3.67- | (0.35- | | | | | | |
| | | 7.97) | 26.07) | 103.10) | 388.50) | | | | | | |
| IL-10 | 0.163 | 1.28 | 1.54 | 8.60 | 2.47 | >0.9999 | 0.2226 | 0.9612 | 0.7382 | >0.9999 | >0.9999 |
| | | (0.89- | (0.53- | (4.53- | (0.86- | | | | | | |
| | | 2.71) | 20.99) | 20.95) | 20.65) | | | | | | |
| TNFα | 0.1807 | 10.80 | 14.42 | 16.46 | 17.50 | >0.9999 | 0.3852 | 0.3386 | >0.9999 | >0.9999 | >0.9999 |
| | | (7.52- | (12.11- | (13.80- | (13.58- | | | | | | |
| | | 16.72) | 18.98) | 24.55) | 28.08) | | | | | | |
| IL-7 | 0.1915 | 4.02 | 5.06 | 6.77 | 7.97 | >0.9999 | >0.9999 | 0.6851 | >0.9999 | 0.5046 | >0.9999 |
| | | (1.96- | (3.19- | (3.55- | (5.01- | | | | | | |
| | | 10.35) | 6.53) | 11.41) | 14.94) | | | | | | |
| MDC/ | 0.2003 | 1860 | 1413 | 672 | 980 | >0.9999 | 0.4423 | >0.9999 | 0.4992 | >0.9999 | >0.9999 |
| CCL22 | | (804- | (904- | (544- | (664- | | | | | | |
| | | 2542) | 1811) | 1531) | 2842) | | | | | | |
| IP-10/ | 0.2277 | 793 | 442 | 598 | 600 | 0.5512 | >0.9999 | >0.9999 | >0.9999 | 0.3084 | >0.9999 |
| CXCL10 | | (447- | (244- | (406- | (463- | | | | | | |
| | | 1362) | 685) | 1234) | 928) | | | | | | |
| IFNα2 | 0.2493 | 8.00 | 23.06 | 47.36 | 36.91 | >0.9999 | >0.9999 | 0.9474 | 0.9841 | 0.8055 | >0.9999 |
| | | (8.00- | (8.75- | (21.80- | (11.10- | | | | | | |
| | | | 49.08) | 80.65) | 108.2) | | | | | | |
| FGF2 | 0.2967 | 58.12 | 52.48 | 106.2 | 62.95 | >0.9999 | 0.9468 | >0.9999 | 0.7486 | >0.9999 | 0.6147 |
| | | (40.06- | (28.65- | (65.28- | (32.10- | | | | | | |
| | | 104.30) | 116.90) | 191.50) | 113.60) | | | | | | |
| IL-17A | 0.299 | 1.48 | 4.67 | 5.73 | 2.53 | >0.9999 | 0.7904 | >0.9999 | >0.9999 | >0.9999 | 0.515 |
| | | (0.95- | (1.52- | (2.67- | (0.80- | | | | | | |
| | | 5.78) | 6.58) | 16.84) | 8.12) | | | | | | |
| IL-9 | 0.3033 | 0.09 | 0.12 | 3.53 | 0.22 | 0.9181 | 0.3555 | >0.9999 | >0.9999 | >0.9999 | >0.9999 |
| | | (0.09- | (0.12- | (0.0.20- | (0.09- | | | | | | |
| | | 0.33) | 9.96) | 11.28) | 9.04) | | | | | | |
| G-CSF | 0.3325 | 8.59 | 15.40 | 28.17 | 18.67 | >0.9999 | 0.5804 | >0.9999 | >0.9999 | >0.9999 | >0.9999 |
| | | (2.75- | (8.15- | (9.41- | (4.80- | | | | | | |
| | | 23.86) | 19.30) | 81.34) | 48.59) | | | | | | |
| GM-CSF | 0.3370 | 5.71 | 3.64 | 14.25 | 5.93 | >0.9999 | 0.8835 | >0.9999 | 0.7246 | >0.9999 | >0.9999 |
| | | (1.87- | (2.28- | (4.85- | (3.16- | | | | | | |
| | | 7.61) | 14.12) | 22.72) | 16.39) | | | | | | |
| MIP-Iβ/ | 0.3894 | 28.58 | 41.85 | 46.87 | 56.15 | >0.9999 | >0.9999 | 0.771 | >0.9999 | >0.9999 | >0.9999 |
| CCL4 | | (7.69- | (19.47- | (27.65- | (22.36- | | | | | | |
| | | 51.94) | 59.22) | 82.03) | 113.5) | | | | | | |
| IL-5 | 0.4140 | 0.75 | 0.87 | 3.13 | 0.88 | >0.9999 | >0.9999 | >0.9999 | >0.9999 | >0.9999 | 0.9388 |
| | | (0.49- | (0.69- | (0.85- | (0.52- | | | | | | |
| | | 1.83) | 1.19) | 9.32) | 11.48) | | | | | | |
| IL-2 | 0.5540 | 1.52 | 1.66 | 2.78 | 1.57 | >0.9999 | >0.9999 | >0.9999 | >0.9999 | >0.9999 | >0.9999 |
| | | (0.84- | (0.97- | (1.29- | (0.63- | | | | | | |
| | | 2.85) | 2.90) | 5.57) | 4.06) | | | | | | |
| IL-3 | 0.671 | 0.69 | 1.21 | 0.82 | 1.22 | >0.9999 | >0.9999 | >0.9999 | >0.9999 | >0.9999 | >0.9999 |
| | | (0.39- | (0.79- | (0.57- | (0.13- | | | | | | |
| | | 1.08) | 1.37) | 1.52) | 2.20) | | | | | | |
| Groα/ | 0.6864 | 1698 | 892 | 756 | 1143 | >0.9999 | >0.9999 | >0.9999 | >0.9999 | >().9999 | >0.9999 |
| CXCL1 | | (890- | (439- | (530- | (447- | | | | | | |
| | | 2065) | 1671) | 1257) | 2709) | | | | | | |
| IL- | 0.9800 | 2.87 | 3.29 | 2.77 | 2.87 | >0.9999 | >0.9999 | >0.9999 | >0.9999 | >0.9999 | >0.9999 |
| 12p70 | | (1.29- | (1.22- | (1.40- | (1.61- | | | | | | |
| | | 9.94) | 6.30) | 9.80) | 8.42) | | | | | | |
| Flt-3L | 0.9968 | 0.39 | 2.73 | 1.60 | 9.49 | >0.9999 | >0.9999 | >0.9999 | >0.9999 | >0.9999 | >0.9999 |
| | | (0.39- | (1.60- | (0.39- | 0.39- | | | | | | |
| | | 63.33 | 5.12) | 40.63) | 29.76 | | | | | | |

### Circulating Soluble Factors are Influenced by Age and Disease Duration to Varying Degrees:

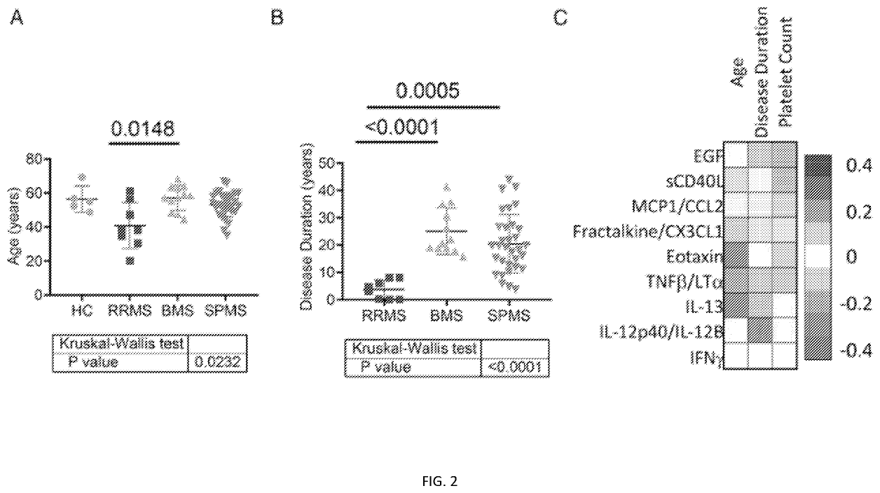

When comparing SPMS to RRMS, RRMS subjects were significantly younger (FIG. 2A) and had a shorter disease duration (FIG. 2B). Therefore, this study took a unique approach by comparing nonprogressive benign MS (BMS), naturally age- and disease duration-matched, to progressive SPMS to dissect progression-specific mechanisms (FIGS. 2A and 2B). Spearman correlation analysis less than 0.3 is considered statistically negligible (FIG. 2C). The analysis indicated that Eotaxin (r=0.3398, 95% CI: 0.0791-0.5570, P=0.0097), TNFβ/LTα (r=0.3133, 95% CI: 0.0495-0.5362, P=0.0177), and IL-13 (r=0.3383, 95% CI: 0.0774-0.5559, P=0.0101) were moderately associated with age, while IL-12p40/IL-12 was moderately associated with disease duration (r=0.3339, 95% CI: 0.0589-0.5618, P=0.0156. Other factors are not significantly associated with age and disease duration. Analysis of plasma levels of EGF and sCD40L did not show a noticeable association with platelet count. EGF can be derived from multiple cellular sources, which may have disparate effects, and levels may be influenced by comorbidities, suggesting a lack of specificity for MS.

Plasma Levels of MCP1/CCL2 and sCD40L are Elevated in SPMS as Compared to RRMS:

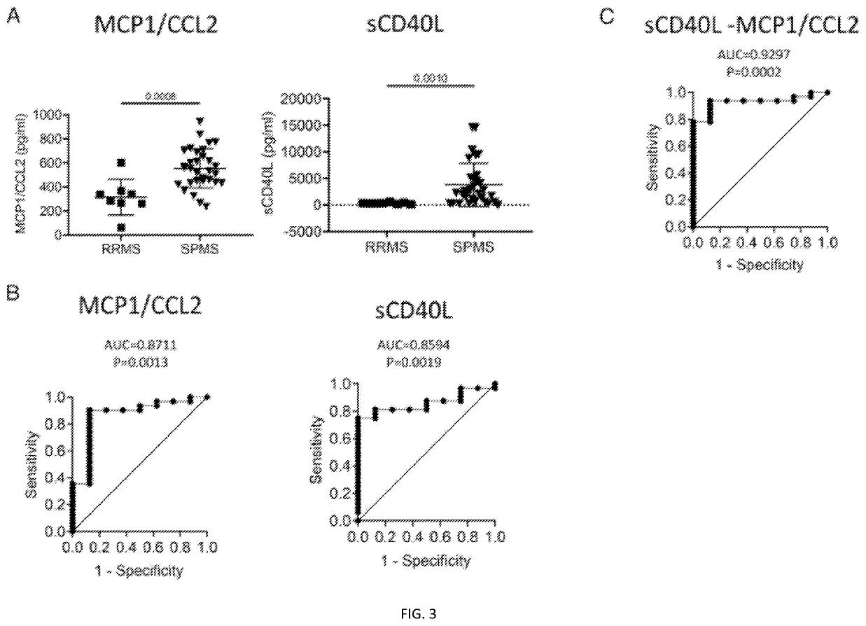

To evaluate whether any of the soluble factors can be used to discriminate SPMS from RRMS, receiver-operating characteristic (ROC) curve analyses were performed (Table 4). Plasma levels of MCP1/CCL2 and sCD40L were significantly increased in SPMS patients compared with RRMS patients (FIG. 3A). ROC analysis revealed that MCP1/CCL2 (AUC=0.8711±0.0762, 95% CI: 0.7218-1.000, P=0.0013) and sCD40L (AUC=0.8594±0.0577, 95% CI: 0.7462-0.9725, P=0.0019) were the best biomarkers to stratify SPMS from RRMS (FIG. 3B). Multiple logistic regression analysis combining plasma levels of MCP/CCL2 and sCD40L gave a higher predictive power (AUC=0.9297±0.04193, 95% CI: 0.8475-1.000, P=0.0002, FIG. 3C), which gave a negative predictive power of 77.78% and a positive predictive power of 96.77% to discriminate SPMS from RRMS.

### Plasma Levels of MCP1/CCL2 are Elevated in BMS as Compared to RRMS:

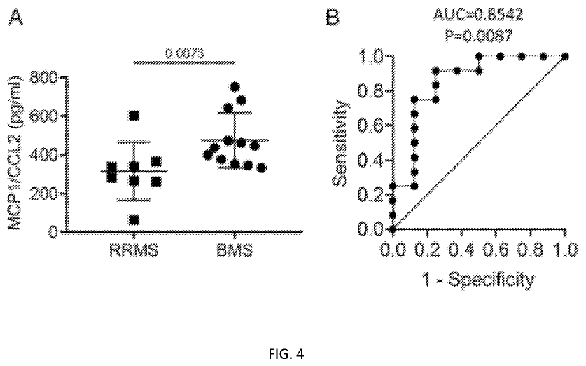

Plasma levels of MCP1/CCL2 were significantly increased in BMS compared with RRMS (P=0.0073, FIG. 4A). ROC curve analysis indicated that MCP1/CCL2 was the best classifier to discriminate BMS from RRMS (AUC=0.8542±0.0965, 95% CI: 0.6651-1.000, P=. 0087, FIG. 4), which gave a sensitivity of 75% and a specificity of 87.5% (Table 4).

TABLE 4
Circulating biomarkers that can discriminate among different MS Types

| | | | | | | |
|---|---|---|---|---|---|---|
| | | p | Cut off | | | Likelihood |
| AUC | 95% Cl | Value | (pg/ml) | Sensitivity | Specificity | Ratio |

RRMS vs SPMS

| | | | | | | | |
|---|---|---|---|---|---|---|---|
| MCP1/CCL2 | 0.8711 | 0.7218 to 1.000 | 0.0013 | >369.4 | 90.63 | 87.5 | 7.25 |
| sCD40L | 0.8594 | 0.7462 to 0.9725 | 0.0019 | >487.6 | 81.25 | 87.5 | 6.50 |
| MCP1/CCL2 | 0.9297 | 0.8475 to 1.000 | 0.0002 | NA | NA | NA | NA |
| & sCD40L | | | | | | | |

RRMS vs BMS

| | | | | | | | |
|---|---|---|---|---|---|---|---|
| MCP1/CCL2 | 0.8542 | 0.6651 to 1.000 | 0.0087 | 371.9 | 75 | 87.5 | 6.0 |

BMS VS SPMS

| | | | | | | | |
|---|---|---|---|---|---|---|---|
| sCD40L | 0.7865 | 0.6408 to 0.9321 | 0.0037 | >2206 | 53.13 | 91.67 | 6.38 |
| IFN-γ | 0.7109 | 0.5575 to 0.8644 | 0.0328 | <7.805 | 56.25 | 83.33 | 3.38 |
| sCD40L & | 0.8307 | 0.7078 to 0.9536 | 0.0008 | NA | NA | NA | NA |
| IFNγ | | | | | | | |

ROC curve analysis and multiple logistic regression analysis were performed using the plasma level of these soluble factors from RRMS (n = 8), BMS (n = 12), and SPMS (n = 32), AUC: under curve value, 95% CI: 95% confidence interval.

Plasma Levels of sCD40L and IFN-γ Differentiate Nonprogressive BMS from SPMS:

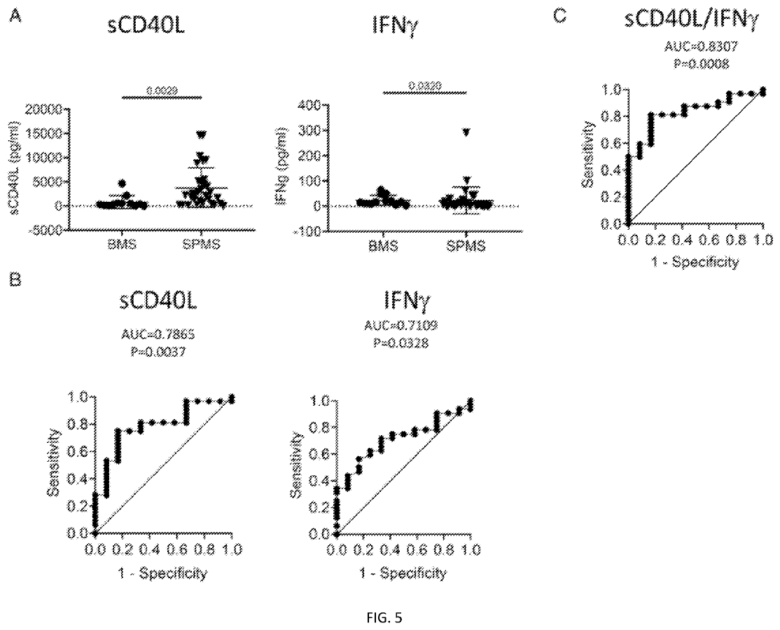

Comparison of non-progressive BMS and SPMS is the most informative analysis to reveal underlying mechanisms of MS disease progression given that these groups typically have similar age and disease duration but different disease progression rates. Plasma levels of sCD40L detected by Luminex assay revealed significant elevation in patients with SPMS compared to those with BMS (P=0.0029; FIG. 5A). Mann-Whitney analysis comparing the BMS group and the SPMS group also revealed IFNγ significantly discriminates between non-progressive BMS and SPMS (P=0.0320, FIG. 5A). ROC analysis revealed that sCD40L (AUC=0.7865±0.0743, 95% CI: 0.6408-0.9321, P=0.0037) and IFNγ (AUC=0.7109±0.07829, 95% CI: 0.5575-0.8644, P=. 0328) could discriminate SPMS from BMS (FIG. 5B). Multiple logistic regression analysis showed that combining IFNγ with sCD40L improved the stratification (AUC=0.8307±0.0627, 95% CI: 0.7078-0.9536, P=0.0008, FIG. 5C), which gave a negative predictive power of 60.00 and a positive predictive power of 82.35% to discriminate BMS and SPMS.

MCP1/CCL2 and sCD40L are Positively Correlated MS Disease Progression:

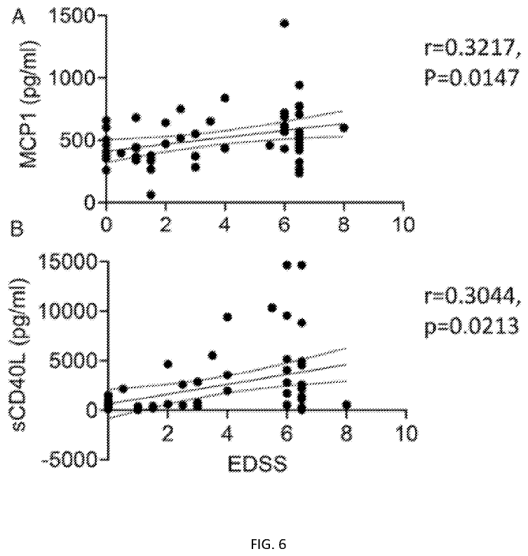

Spearman correlation analysis was used to determine whether any of the plasma soluble factors are linked to MS progression across all cohorts using EDSS as a disease progression marker. Only MCP1/CCL2 (r=0.3217, 95% CI: 0.05890-0.5428, P=0.0147, FIG. 6A) and sCD40L (r=0.3044, 95% CI: 0.03975-0.5292, P=0.0213, FIG. 6B) showed positive correlation with EDSS, suggesting that their upregulation is associated with MS disease progression. It is unclear whether these factors play a causative role in MS progression or reflect the body's physiological response directly or indirectly to MS progression.

### Correlations of Inflammatory Cytokines are SPMS-Specific:

To find out whether any of the plasma soluble factors are correlated with each other specifically in SPMS, a comparative correlation analysis was conducted using all of the factors analyzed in this study to compare the SPMS group with the non-SPMS group including BMS/RRMS. Significant positive correlations were found among the majority of cytokines for SPMS groups, suggesting a shift to a global proinflammatory innate immune environment in SPMS patients. Notably, there was a significant correlation of collectively elevated cluster of soluble factors only with SPMS, which includes sCD40L, Eotaxin, EGF, and MCP1/CCL2. sCD40L showed a significant positive correlation with EGF (r=0.6087, P=0.0002), Eotaxin (r=. 5943, P=0.0003), as well as MCP1/CCL2, although less significantly (r=0.4713, P=0.0065) in the SPMS group of patients. However, their correlations in HC, RRMS, and BMS were limited and insignificant. EGF seemed to show a significant correlation with many other cytokines in non-SPMS. This study suggests that sCD40L, along with Eotaxin/CCL11 and MCP/CCL2, appears to have a close association with MS progression into the SPMS stage and could play an important role in the pathogenic process of MS progression. Therefore, they could be potential biomarkers and therapeutic targets for SPMS.

Neurofilament Light Chain (NFL) Levels Correlate with sCD40L Levels and are Indicative of Disease Progression

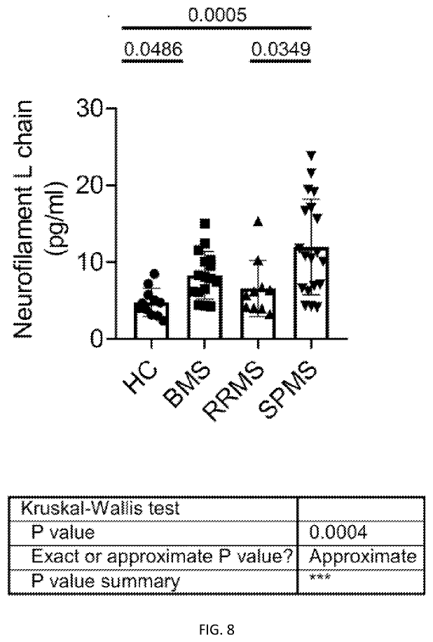

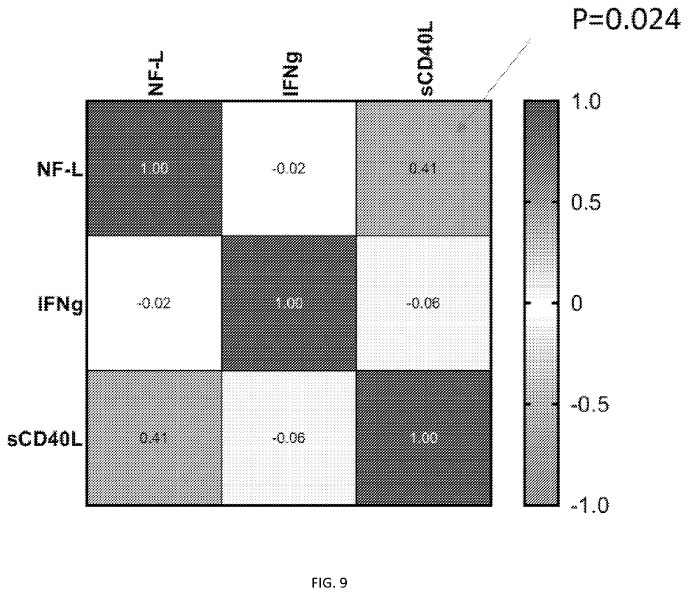

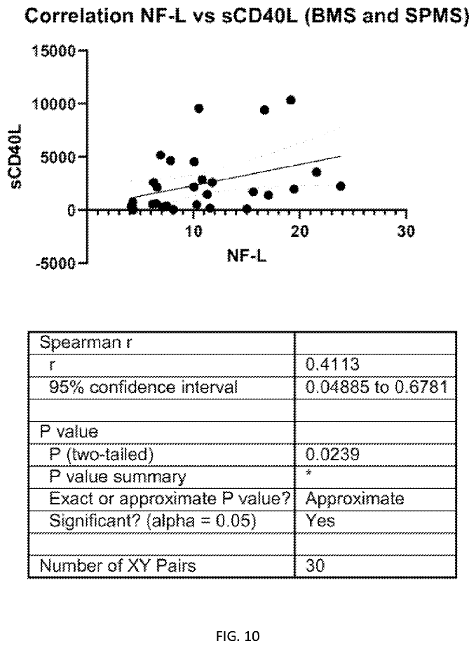

NFL levels were also measured in control subjects, subjects with BMS, subjects with RRMS, and subjects with SPMS. Results are shown in FIG. 8. NFL levels are significantly higher in subjects having SPMS compared to controls (p=0.0005) and significantly higher in subjects having SPMS compared to subjects having RRMS (p=0.0349). NFL levels are also significantly higher in subjects having BMS compared to controls (p=0.0486). Taken together, this data indicates that NFL levels are indicative of BMS compared to a control (e.g. non-diseased) state, and that increased NFL levels are indicative of progression from RRMS to SPMS. As shown in FIG. 9, NFL levels were also found to correlate with sCD40L levels in patients with MS. As shown in FIG. 10, NFL levels correlate with sCD40L levels to predict progression of MS, where elevated levels of NFL and sCD40L are indicative of SPMS, and lower levels of NFL and sCD40L are indicative of BMS.

### Discussion

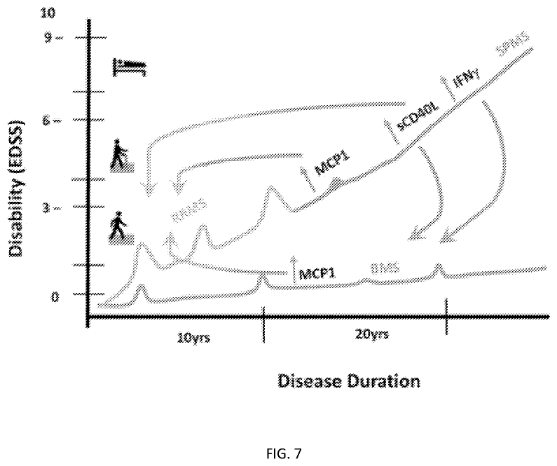

The lack of effective therapies for progressive MS makes the identification of progression-related factors crucial. The technology provided herein was derived from taking a novel approach by comparing age- and disease duration-matched non-progressive BMS to SPMS. Notable plasma marker differences were found between non-progressive BMS and SPS that may be used as potential biomarkers specific for MS disease progression (FIG. 7). The combined sCD40L and IFNγ levels distinguish between non-progressive BMS and SPMS with AUC=0.8307 (P=0.0008) in the multiple logistic regression analysis (FIGS. 5 and 6).

The role of epidermal growth factor (EGF) in MS remains controversial because it is associated with not only the growth and proliferation of CNS cells (e.g., neurons, astrocytes, and oligodendrocytes) but also neurotoxic immune responses. Although EGF was identified as the top differential cytokine across cohorts, the secretion of EGF by various cell types complicates the interpretation of these differences. Circulating EGF levels have been implicated as a prognostic marker for other neurodegenerative diseases, and EGF dysregulation is also associated with kidney disease, suggesting that changes in EGF are not specific for MS and that levels may be influenced by the presence of comorbidities. Consequently, circulating EGF levels may be highly influenced by patient characteristics as well as heterogeneity within and across cohorts.

### Role of MCP1/CCL2, Fractalkine/CX3CL1, and Eotaxin-1/CCL11 in MS Disease Progression:

Chemokines mediate the migration of immune cells into the CNS. MCP1/CCL2 is a monocyte chemoattractant that influences migration of monocytes, activated T cells, and macrophages across the endothelium of blood vessels. Both Fractalkine/CX3CL1 and Eotaxin-1/CCL11 showed significant age effects in the analysis presented herein. Fractalkine/CX3CL1 is a chemokine exhibiting chemoattractant effects on effector cells with cytotoxic function in the membrane-bound form, including natural killer (NK) cells and cytotoxic T cells. In the CNS, the interaction between fractalkine/CX3CL1 and its corresponding receptor (CX3CR1) modulates microglial activation. The results herein indicate that soluble fractalkine/CX3CL1 levels tend to be higher in BMS than SPMS or RRMS. Furthermore, the decrease in soluble fractalkine levels seen with age may enhance neuroinflammatory processes that promote neurodegeneration in the CNS.

Eotaxin-1/CCL11 is an eosinophil recruiting chemokine in allergic diseases. This study shows that serum and plasma levels of Eotaxin-1/CCL11 are increased in SPMS compared to RRMS. The progression from RRMS to SPMS may occur in older adults as circulating levels of Eotaxin-1/CCL11 increase. The increase in Eotaxin-1/CCL11 levels with age may then contribute to the neurodegenerative mechanisms of progression and promote the transition from RRMS to SPMS.

Taken together, the results show that sCD40L finds use as a biomarker associated with disease progression in MS. CD40/CD40L finds use as a therapeutic target to control MS progression. In addition to sCD40L, this study also recognizes the important role that MCP1/CCL2 and IFNγ plays in different stages of MS progression (FIG. 7). With better stratification of MS patient populations, clinical trials focused on actively progressing MS can be more effectively carried out, leading to improved treatments for these patients.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

Any patents and publications referenced herein are herein incorporated by reference in their entireties.

1.-52. (canceled)

53. A method comprising measuring protein expression levels of at least 6 proteins in a sample obtained from a subject having or suspected of having multiple sclerosis, wherein the at least 6 proteins are selected from soluble CD40L (sCD40L), Monocyte Chemoattractant Protein-1 (MCP-1), gamma interferon (IFNγ) epidermal growth factor (EGF), neurofilament light chain (NFL), fractalkine/CX3CL1, interleukin-13 (IL-13), Eotaxin, lymphotoxin-alpha (TNFβ/LTα), and interleukin-12 subunit p40 (IL-12p40).

54. The method of claim 53, wherein the at least 6 proteins comprise at least one of sCD40L, MCP-1, IFNγ, and NFL.

55. The method of claim 53, wherein the at least 6 proteins comprise at least two of sCD40L, MCP-1, IFNγ, and NFL.

56. The method of claim 53, wherein the at least 6 proteins comprise at least three of sCD40L, MCP-1, IFNγ, and NFL.

57. The method of claim 53, wherein the at least 6 proteins comprise each of sCD40L, MCP-1, IFNγ, and NFL.

58. The method of claim 53, comprising measuring protein expression levels of at least 7 proteins in the sample.

59. The method of claim 53, comprising measuring protein expression levels of at least 8 proteins in the sample.

60. The method of claim 53, comprising measuring protein expression levels of at least 9 proteins in the sample.

61. The method of claim 53, wherein the protein expression levels are measured by an immunoassay.

62. The method of claim 53, wherein the sample is a plasma sample.

63. The method of claim 53, further comprising:
a) identifying the subject as having relapsing-remitting multiple sclerosis based on the protein expression levels of the at least 6 proteins, and treating the subject identified as having relapsing-remitting multiple sclerosis with a disease modifying therapy; or
b) identifying the subject as having secondary progressive multiple sclerosis based on the protein expression levels of the at least 6 proteins, and treating the subject identified as having secondary progressive multiple sclerosis with one or more therapies selected from stem cell therapy, steroids, beta interferon, glatiramer acetate, cladribine, dimethyl fumarate, and Siponimod.

64. The method of claim 53, wherein the subject is a human.
